SEQUENCE LISTING (11 sequences)

SEQ ID NO: 1 - protein, 469 aa, Unknown
  Derived from metagenome
MMGGVGVREELATWTAVRLAEHIRKKELSPVEVTDYFLRRIEALNPAVNAFCTVDADGAMRAAKAAEQRLMAGETPPLLGVPVAIKDLTPTKGIRTTYGSRLFADNVPEADAVLVTRLKQAGAIIVGKTNTPEFGHAGVTDNRLFGRTNNPWDLSRIAGGSSGGSDGGGSIRIPASCCGIFGFKPTFGRVPHDTGATAFSITAPFLHHGPMSRTVEDSVLMLAAMQGPDGCDPFSLPLPGIDWPLSAEIKPFSQWRIAYSPNLDFYAIDPAVRQVMEQAVSALQGLGCRVEEVRLGLEEGKTLVLETFARLWAVHYAAFYEELLEREAELSKGFVATIRYGQQFSAVEYKRLERPRAVVYERVENVFAKYDLLITPTLAVPPFAHDCPPREIDGKAVNPYNEWMLTSIFNLTGHPVASIPAGFSPEGLPIGMQIVGPRLADAAVLEFAYLFEQTVAPRRPYPCDDVRLN

SEQ ID NO: 2 - protein, 264 aa, Unknown
  Derived from metagenome
  misc_feature (252)..(252): Xaa can be any naturally occurring amino acid
LDYLGGFSPLESDVTVEKTRIAGVPGEWISTPDARKDRVLFYLHGGAYCFGSCDSHRGLVSRLARACGSRALLIEYRLAPEHPFPAALEDSTAAYRELIRSGVRPENLVIAGDSAGGGLTMATLLTLRDEGDPLPSAAVLLSPWTDLEGTGESMKTKADVEPWLDPEKSHLLAKLYLGDLDPRHPLVSPIHADLNNLPPLLVHVGSDECLLDDSVRLVERAKSAGVETEFKICDEMWHVFHGFPIPEAQQAXEEIGAFVRARLP

SEQ ID NO: 3 - protein, 297 aa, Unknown
  Derived from metagenome
MASPQSEAIRQMLREQKEAAKKGAPSIEEQRRQLDYLGGFSPLESDVTVEKTRIAGVPGEWISTPDARKDRVLFYLHGGAYCFGSCDSHRGLVSRLARACGSRALLIEYRLAPEHPFPAALEDSTAAYRELIRSGVRPENLVIAGDSAGGGLTMATLLTLRDEGDPLPSAAVLLSPWTDLEGTGESMKTKADVEPWLDPEKSHLLAKLYLGDLDPRHPLVSPIHADLNNLPPLLVHVGSDECLLDDSVRLVERAKSAGVETEFKIWDEMWHVFHGFPIPEAQQAIEEIGAFVRARLP

SEQ ID NO: 4 - protein, 311 aa, Unknown
  Derived from metagenome
MADPQLEAVLVGLAQASAGAQGPATVEGFRVALRELTRMLDFRDIPVGRVENRMIPGPDGEIGIRIYTPIAAGARMLETLIYFHGGGFVAGDLETHDTLCRGLTARSGCRVISVDYRLAPEHPFPAAIDDSYAALRWIEANATTLGVDSNRIAVGGDSAGGNIAAVVAQLARGAGNPVVRFQLLIYPVVQWDVATPSRQQFAEDPIIPRDVIDMCARNYFGPMVPATDFRAAPLAASDLAGLPPAYVITAGLDPLRDEGAQYAEKLREAGVAVEHVGYDDMIHGFMSMSNALDTAKLAIERAGDALRNALR

SEQ ID NO: 5 - protein, 316 aa, Unknown
  Derived from metagenome
MSLDPKARELLAMVYRVNAPRFHELSVSQARHATQKLMFAFRPEAPAVASTTEVPIPRPDGSVLFARLYRPLGCHASEDLGLLIYFHGGGWCTGDLPGYDVLCRELANQSGAAVLSVDYRLAPEHRFPAAVHDASLAFEWSTENASLLGVDAERIALGGDSAGGNLAIVAALEARDRAARMPRALALIYPSTQIHSERSSRETFADGYFLDRESLRWFYEHYFADPAQAQSWQASPMLAASLAGLPPAILITAGCDPLTDDCVAFAERMVADGGLVVRHHFEGMVHGFLPLGKFFAQANEAVRCVSSYLREALQAS

SEQ ID NO: 6 - protein, 297 aa, Unknown
  Derived from metagenome
  misc_feature (216)..(216): Xaa can be any naturally occurring amino acid
MSLEELAVVRQLLAGLVTGEARSLEDFRTSYDEAGKAFGLPEGVTVTPVSAGGVPGEWLAPAAGAGKRVLLYLHGGGYALGSLDSHRHLAAHTALALNGRVLLIDYRRSPEHPFPAAVDDALAAYRWLTETGVDPAKLAVAGDSAGGGLTVAVLLAARDAGLRLPAAAVCISPWANLENKGASYGAKANVDPMVRHADLELWTAAYLGTSTPRRAXLASPVYADLNGLPPFLIQVGSSEVLLSDSHLLADRLKEAGVSVDLHVWPEMIHVWHWFAPVLSEGRAAIDEMASFLDTKLG

SEQ ID NO: 7 - protein, 490 aa, Unknown
  Derived from metagenome
MTGLHFRSASELGRMIRRGEISSAELTDHFIQRIETLDGKTNAVVARDFDRARALAKEADAAQARGASLGALHGLPFTIKDAYEVEGIVSTGGNPTWKDHVPTSSATAVERLQRSGAIVMGKTNVPYLSGDLQTYNDIYGTTNNPWALDCGPGGSSGGSAASLAAGFAAAEFGSDIGGSIRTPAHLCGVFGHKPSFGIVPKRGHLSPPPGCLSEGDLSVAGPLARSAEDLKLLLSLTAGPDWADAAGWKLDLPPARARTPRELRAAVWIDDEFCDIDRESADLLRNAAKALQDAGANVDWNARPDFTLAEITECYLILLHSQIGAGMPQSIRDHWAEMKKGFAPDDKSHAALQAIGGTLSLAERAVWKEVQAQLRWKWHTFFKSYDVVLSPVLMRPAFEHNHQSNWHKRELDVNGVKRPYMDVLIWAGPAVVSYLPATAAPVGVTSEGKPVGIQIIGPHLEDYTTIAVAGMFEEILGGFKPPKGWAAALE

SEQ ID NO: 8 - protein, 177 aa, Unknown
  Derived from metagenome
CACCLSLVDRDGRRPGELAVAGDSAGGGLTVAVLLAARDAGLRLPAAAVCISPWANLENKGASYGAKANVDPMVRHADLELWTAAYLGTSTPRRAPLASPVYADLNGLPPFLIQVGSSEVLLSDSHLLADRLKEAGVSVDLHVWPEMIHVWHWFAPVLSEGRAAIDEMASFLDTKLG

SEQ ID NO: 9 - protein, 491 aa, Unknown
  Derived from metagenome
LERSDLDYASATEIARLVRTRQISAADVTEHAISRIEARNGSLNAFVYTDFEQARSRAKDLDTRISAGEDVGPLAGVPTAIKDLFNFYPGWPSTLGGIRCLRDFKLDVKSRYATKMEEAGAVVLGITNSPVLGFRGTTDNDLYGPTRNPFDLSRNSGGSSGGTSAAVADGLLPIGDGTDGGGSIRIPAAWCHVFGFQASPGRIPLAIRPNAFGAAAPFIYEGPITRTVEDAALAMSVLAGSDPADPFSLNDRLDWLGAVDQPITSLRIGFTPDFGGFPVEPAVAATIAHAVRAFEQAGAKIVPLKLDFGYTHDELSQLWCRMISQGTIAVVDSFAENGLHLEPDFPAPVMEWAQKAKNATPLDLHRDQVMRTKVYDVLNAAFSQVDLIAGPTTTCLPTPNGERGMTVGPSEIAGTPINRLIGFCPTFLTNFTGNPAASLPAGLADGLPVGLMLIGPRRDDLTVLSASAAFERVQPWADSYRIPAARPLGSQ

SEQ ID NO: 10 - protein, 330 aa, Unknown
  Derived from metagenome
MRPRSRPHARARGAPTILRDPATMALHRTPRRNDMADRGIEVVHAHLAKLPPADSLTVAERRAQYERAEKVFPLSPDVKVERVTAGAAPAEWLRPPSARAGHVVLYLHGGGYVIGSPRSHRHLAAAIAGAAGTNALLLDYRLAPEHPFPAALDDAVAAYRWLLDQGIAAEHIAVAGDSAGGGLTVATLLALRDAHLPRPAAGVCISPWVDLTCSGGSYQSKAGVDPIVRQAGVAEMARAYLGATDPRSPLASPLFADLRGLPPLLIHVGSDEVLLDDAIGLAERAKAAGVDATLEQWDRMIHVWHWFLPMLDEAQTAVESIGRFVRARTA

SEQ ID NO: 11 - protein, 544 aa, Pig
GQPASPPVVDTAQGRVLGKYVSLEGLAQPVAVFLGVPFAKPPLGSLRFAPPQPAEPWSFVKNTTSYPPMCCQDPVAGQMTSDLFTNRKERLIPEFSEDCLYLNIYTPADLTKRGRLPVMVWIHGGGLVVGGASTYDGLALAAHENVVVVAIQYRLGIWGFFSTGDEHSRGNWGHLDQVAALHWVQENIANFGGDPGSVTIFGESAGGESVSVLVLSPLAKNLFHRAISESGVAFTAGLVRKDMKAAAKQIAVLAGCKTTTSAVFVHCLRQKSEDELLDLTLKMKFFALDLHGDPRESHPFLTTVVDGVLLPKMPEEILAEKDFNTVPYIVGINKQEFGWLLPTMMGFPLSEGKLDQKTATSLLWKSYPIANIPEELTPVATDKYLGGTDDPVKKKDLFLDLMGDVVFGVPSVTVARQHRDAGAPTYMYEFQYRPSFSSDKKPKTVIGDHGDEIFSVFGAPFLRGDAPEEEVSLSKTVMKFWANFARSGNPNGEGLPHWPMYDQEEGYLQIGVNTQAAKRLKGEEVAFWNDLLSKEAAKKPPKIK

(19) United States
(12) Patent Application Publication
(10) Pub. No.: US 2025/0034606 A1
(43) Pub. Date: Jan. 30, 2025

(54) METHOD FOR CLEAVING POLYURETHANE PRODUCTS

(71) Applicant: Covestro Deutschland AG, Leverkusen (DE)

(72) Inventors: Dirk Hinzmann, Pulheim (DE); Verena Schiffer, Bedburg (DE); Antje Wehlau, Leverkusen (DE); Lena Schaffert, Köln (DE); Lukas Reisky, Köln (DE)

(21) Appl. No.: 18/567,935

(22) PCT Filed: Jun. 13, 2022
(86) PCT No.: PCT/EP2022/065933

§ 371 Date: Dec. 7, 2023

(30) Foreign Application Priority Data
Jun. 14, 2021 (EP) 21179237.9
May 6, 2022 (EP) 22171964.4

(51) Int. Cl. C12P 13/00; C08J 11/24; C12N 9/80; C12P 13/02

(52) U.S. Cl. CPC C12P 13/001; C08J 11/24; C12N 9/80; C12P 13/02; C12Y 305/01075; C08J 2375/04

(57) ABSTRACT

The invention relates to a method for cleaving polyurethane products, having the steps of (A) providing a polyurethane product which is based on isocyanate components and a polyol component; (B) reacting the polyurethane product with a monofunctional araliphatic alcohol in the presence of an alcoholysis catalyst, thereby obtaining a product mixture containing a liquid polyol phase and a solid carbamate of an isocyanate of the isocyanate component and the monofunctional araliphatic alcohol; and (C) separating the carbamate from the product mixture, thereby leaving the liquid polyol phase.

The present invention relates to a process for cleavage of polyurethane products comprising the steps of: (A) providing a polyurethane product based on an isocyanate component and a polyol component; (B) reacting the polyurethane product with a monofunctional araliphatic alcohol in the presence of an alcoholysis catalyst to obtain a product mixture containing (i) a liquid polyol phase and (ii) a solid carbamate of an isocyanate of the isocyanate component and of the monofunctional araliphatic alcohol; and (C) separating the carbamate from the product mixture, leaving behind the liquid polyol phase. The process according to the invention allows the recovery of raw materials upon which the polyurethane product is based. Polyols may especially be recovered from the polyol phase and the separated carbamate may be cleaved into an isocyanate of the isocyanate component (thermal, optionally catalytically-assisted carbamate cleavage) or converted into the corresponding amine (hydrolysis or hydrogenolysis).

Polyurethane products enjoy a diversity of applications in industry and in everyday life. Distinctions are typically made between polyurethane foams and what are known as “CASE” products, with “CASE” being a collective term for polyurethane coatings (e.g., paints), adhesives, sealants and elastomers. The polyurethane foams are typically divided into rigid foams and flexible foams. Common to all of these products in spite of their heterogeneity is the basic polyurethane structure, which is formed by the polyaddition reaction of a polyfunctional isocyanate and of a polyol and which in the case, for example, of a polyurethane based on a diisocyanate O═C═N—R—N═C═O and a diol H—O—R′—O—H (where R and R′ denote organic radicals) may be represented as

˜˜˜[O—R′—O—(O═C)—HN—R—NH—(C═O)]˜˜˜.

It is precisely the great economic success of polyurethane products that is responsible for the large amounts of polyurethane waste generated (for example from old mattresses or seating furniture) that must be sent to a sensible use. The mode of reuse that is the easiest to implement technically is that of incineration, with the heat of combustion released being utilized for other processes, examples being industrial processes. However, this does not allow raw material loops to be closed. Another mode of reuse is so-called “physical recycling”, which sees polyurethane wastes mechanically comminuted and used in the production of new products. This type of recycling naturally has limits and there has therefore been no lack of attempts to recover the basic raw materials of polyurethane production by retrocleavage of the polyurethane bonds (so-called “chemical recycling”). These raw materials to be recovered comprise primarily polyols (i.e., in the example above, H—O—R′—O—H). In addition, it is also possible through hydrolytic cleavage of the urethane bond to recover amines (i.e., in the example above, H₂N—R—NH₂) which can be phosgenated to afford isocyanates (in the aforementioned example to afford O═C═N—R—N═C═O) after workup.

A variety of chemical recycling approaches have been developed in the past. The three most important are briefly summarized as follows:
1. Hydrolysis of urethanes by reaction with water to recover amines and polyols with formation of carbon dioxide.
2. Glycolysis of urethanes by reaction with alcohols, wherein the polyols incorporated in the urethane groups are replaced by the alcohol employed and thus liberated. This process is commonly referred to in the literature as transesterification (more accurately: transurethanization). Regardless of the exact nature of the alcohol used, this mode of chemical recycling is dubbed glycolysis in the literature, a term that really applies only for glycol. In the context of the present invention the term alcoholysis is thus generally used. The glycolysis may be followed by a hydrolysis. If the hydrolysis is performed in the presence of the still-unchanged glycolysis mixture, this is referred to as a
3. Hydroglycolysis of urethane bonds by reaction with water and alcohols, where the hydrolysis and glycolysis processes described above run in parallel.

A summary of the known methods of polyurethane recycling is offered by the review article by Simón, Borreguero, Lucas and Rodríguez in Waste Management 2018, 76, 147-171 [1]. The article highlights glycolysis (2. above) as particularly significant. In glycolysis, a differentiation is made between “biphasic” and “monophasic” regimes depending on whether the obtained crude product of the reaction with the alcohol separates into two phases or not. This depends in particular on the choice of alcohol used and the process conditions (especially the proportion of alcohol used in the reaction mixture and temperature). The aforementioned review article favors the biphasic regime using crude glycerol (wastes from biodiesel production for instance) since it is said to have the greatest potential to recover high-quality products at low production costs (wherein recovery of the polyols is clearly the focus).

As a result of the additional use of water the product of hydroglycolyses (3. above) is always biphasic. Braslaw and Gerlock in Ind. Eng. Chem. Process Des. Dev. 1984, 23, 552-557 [2] describes the workup of such a product comprising removal of the water (by laboratory-scale phase separation or by evaporation in a process recommended for industrial scale applications and known as the “Ford Hydroglycolysis Process”) and extraction of the remaining organic phase with hexadecane to form an alcohol phase, from which amine can be recovered, and a hexadecane phase, from which polyol can be recovered. Though mentioning the option of recovering amine, the emphasis in this article too is on recovering polyols. A patent for a process operating on these principles was granted under number U.S. Pat. No. 4,336,406.

DE 10 2006036007 A1 describes a process for retrocleavage of polyurethanes/polyurethane ureas, wherein a) such polymers are initially reacted with secondary aliphatic or secondary cycloaliphatic amines to form secondary bisureas and hydroxyl-comprising diols or polyols and optionally amino-comprising compounds, b) the secondary bisureas are separated from the hydroxyl- or amino-comprising compounds, c) the separated secondary bisureas are cleaved with hydrogen chloride to afford the starting isocyanates, d) the resulting isocyanates are separated from the co-formed HCl salt of the secondary amine and the two products are worked up separately and wherein e) the hydroxyl-comprising/amino-comprising compounds formed during treatment with the secondary aliphatic or cycloaliphatic amine are worked up and purified separately.

EP 1 149 862 A1 describes a process in which a rigid polyurethane foam from a used refrigerator is pulverized, liquefied by glycolysis or aminolysis and subsequently treated with supercritical or non-supercritical water. The crude product thus obtained is fractionated and used in the production of new refrigerators.

Only a few of the chemical recycling processes known from the literature are in sustained operation on an industrial scale; many have not even reached pilot scale [1]. In view of generally increased environmental awareness and increased efforts to configure industrial processes to be as sustainable as possible—both of which are fundamentally in favor of chemical recycling—this shows clearly that the chemical recycling of polyurethane products is still by no means mature from a technical and economic point of view. The majority of publications in this field are concerned with the recovery of polyols, in part also with recovery of amines, upon which the polyurethane product was originally based. One approach for the recovery of the amine is described in WO 2020/260387 A1. Once alcoholysis is complete (for example with diethylene glycol) the obtained process product in its entirety is extracted with a solvent that is incompletely miscible with the alcoholysis alcohol (for example toluene). Phase separation and further workup steps afford a liquid carbamate phase which is hydrolyzed. This is followed by isolation of the resulting amine. In a particularly advantageous embodiment which provides an economic and environmentally friendly outlet for impurities originating from the polyurethane product this is carried out by incorporating the recovery of the amine from the amine phase into the workup of newly produced amine in such a way that the amine phase of a crude product fraction of the amine originating from the new production is admixed.

In chemical recycling, for example, challenges in respect of the purity of the recovered polyols remain. These must meet certain purity requirements in order not to adversely affect foaming characteristics when reused in the production of polyurethane foams. If the recovery of the amines is likewise sought it goes without saying that these too shall be isolable in the highest possible purity. The economic and environmentally friendly separation and disposal of the wide variety of auxiliary and additive substances (catalysts, stabilizers and the like) is, however, also important.

However, the carbamates intermediately occurring in a chemolysis using a chemolysis alcohol are—in addition to the abovementioned polyols and amines—likewise valuable products which warrant isolation (especially in the highest possible yield and purest possible form). This would make it possible to perform a subsequent hydrolysis to afford the amines starting from a material of comparatively high purity, thus also affording the amines in much higher purity than when reaction mixtures from the chemolysis are subjected to the hydrolysis. Isolation of the carbamates also makes it possible to supply them to other intended uses. In light of the abovementioned challenges which typically exist in chemical recycling it is an object of the invention to obtain the carbamates in a form which permits isolation and subsequent targeted reuse.

There is therefore a need for further improvements in the field of chemical recycling of polyurethane products, in particular polyurethane foams. In addition to the objectives of being able to recover polyols and amines from polyurethane products in high purity and efficiently and of having available an economically and ecologically acceptable outlet for the auxiliary and additive substances present in the polyurethane products it would also be desirable to provide a process wherein the carbamates otherwise occurring only as intermediates are obtained in a form allowing their isolation in the purest possible form.

Taking this need into account it is an object of the present invention to provide a process for cleavage of polyurethane products, in particular polyurethane foams, preferably rigid polyurethane foams, in particular for the purpose of recovering raw materials upon which the polyurethane products are based, the process comprising the steps of
(A) providing a polyurethane product based on an isocyanate component and a polyol component;
(B) reacting the polyurethane product with a monofunctional (in respect of hydroxyl groups) araliphatic alcohol in the presence of an alcoholysis catalyst to obtain a product mixture containing a liquid polyol phase and a solid carbamate of an isocyanate of the isocyanate component and of the monofunctional araliphatic alcohol (i.e. a suspension of the carbamate in the polyol phase is present);
and
(C) separating the carbamate from the product mixture, leaving behind the liquid polyol phase.

It has entirely surprisingly been found that the transurethanization of the urethane groups of the polyurethane product by reaction thereof with an araliphatic monofunctional alcohol results in formation of the carbamate in the form of a solid, thus allowing it to be easily separated from the polyol phase.

Polyurethane products in the context of the present invention are the polyaddition products (occasionally also referred to, albeit not entirely correctly, as polycondensation products) of polyfunctional isocyanates (=isocyanate component in the polyurethane preparation) and polyols (=polyol component in the polyurethane preparation). Polyurethane products generally contain, as well as the polyurethane base structure outlined, other structures as well, for example structures having urea bonds. The presence of such structures diverging from the pure polyurethane basic structure in addition to polyurethane structures does not depart from the scope of the present invention. If a blowing agent is employed in the production of the polyurethane product, this results in formation of a polyurethane foam.

The urethane groups may in particular be selected from aromatically or aliphatically bonded urethane groups. In the case of an aromatically attached urethane group, the nitrogen atom is attached directly to an aromatic ring. In the case of an aliphatically attached urethane group, the nitrogen atom is attached to an alkyl radical. The alkyl radical is preferably unbranched and composed of at least one, preferably at least two, and more preferably at least three, carbon atoms.

In the terminology of the present invention the term isocyanates encompasses all isocyanates familiar to those skilled in the art in the context of polyurethane chemistry such as especially tolylene diisocyanate (TDI; the isocyanate corresponding to tolylenediamine, TDA), methylenediphenylene diisocyanate (OCN—C₆H₄—CH₂—C₆H₄—NCO; mMDI, the isocyanate corresponding to methylenediphenylenediamine, mMDA), polymethylenepolyphenylene polyisocyanate (OCN—C₆H₄—CH₂—[C₆H₄ (NCO) CH₂]ₙ—C₆H₄—NCO, wherein n is a natural number of 1 or more, in particular of 1 to 6; pMDI, the isocyanate corresponding to polymethylenpolyphenylenepolyamine, pMDA), a mixture f methylenediphenylene diisocyanate and polymethylenepolyphenylene polyisocyanate (MDI), 1,5-pentane diisocyanate (PDI; the isocyanate corresponding to 1,5-pentanediamine, PDA), 1,6-hexamethylene diisocyanate (HDI; the isocyanate corresponding to 1,6-hexamethylenediamine), isophorone diisocyanate (IPDI; the isocyanate corresponding to isophoronediamine, IPDA) and xylylene diisocyanate (XDI; the isocyanate corresponding to xylylenediamine, XDA). The expression “an isocyanate” naturally also encompasses embodiments in which two or more different isocyanates (e.g. mixtures of MDI and TDI) were used in the production of the polyurethane product, unless explicitly stated otherwise, for instance by the wording “precisely one isocyanate”. The entirety of all isocyanates used in the preparation of the polyurethane product is referred to as isocyanate component (of the polyurethane product). The isocyanate component comprises at least one isocyanate. Analogously, the entirety of all polyols used in the preparation of the polyurethane product is referred to as polyol component (of the polyurethane product). The polyol component comprises at least one polyol.

In the terminology of the present invention the term polyols encompasses all polyols familiar to those skilled in the art in the context of polyurethane chemistry such as, in particular, polyether polyols, polyester polyols, polyether ester polyols, polyacrylate polyols and polyether carbonate polyols or else mixtures of two or more of the abovementioned polyols. The expression “a polyol” naturally also encompasses embodiments in which two or more different polyols were employed in the production of the polyurethane product. Therefore, if reference is made, for example, to “a polyether polyol” (or “a polyester polyol” etc.), this terminology naturally also encompasses embodiments in which two or more different polyether polyols (or two or more different polyester polyols etc.) were employed in the production of the polyurethane product.

Carbamates in the terminology of the present invention are the urethanes formed in step (B) by the reaction with the alcohol.

An amine corresponding to an isocyanate is the amine that can be phosgenated to obtain the isocyanate according to R—NH₂+COCl₂→R—N═C═O+2 HCl. Analogously, a nitro compound corresponding to an amine is the nitro compound that can be reduced to obtain the amine according to R—NO₂+3H₂≥R—NH₂+2H₂O.

In the context of the present invention a monofunctional araliphatic alcohol is to be understood as meaning a compound having a single alcohol group bonded to a carbon atom that is directly bonded to an aromatic group. Examples of monofunctional araliphatic alcohols in this context include benzyl alcohol and furfuryl alcohol.

In the context of the process of the invention the monofunctional araliphatic alcohol is employed in a superstoichiometric amount. This is to be understood as meaning that the alcohol is employed in an amount that is theoretically sufficient to convert all of the polyurethane bonds of the polyurethane product to form carbamates of the alcohol and polyols.

Reported pressures are absolute pressures characterized by a lower case “(abs.)” appended to the pressure unit (generally mbar).

There will initially follow a brief summary of various possible embodiments of the invention:

In a first embodiment of the invention, which may be combined with all other embodiments, the polyurethane product is a polyurethane foam, in particular a rigid polyurethane foam.

In a second embodiment of the invention, which may be combined with all other embodiments, the polyol component comprises a polyether polyol, a polyester polyol, a polyether ester polyol, a polyether carbonate polyol, a polyacrylate polyol or a mixture of two or more of the abovementioned polyols. The polyol component is preferably a polyether polyol. More preferably, the polyol component is a polyether polyol (i.e. does not contain any polyols other than polyether polyols; but a mixture of two or more different polyether polyols is encompassed and does not depart from the scope of this embodiment).

In a third embodiment of the invention, which may be combined with all other embodiments, the isocyanate component comprises tolylene diisocyanate, methylenediphenylene diisocyanate, polymethylenepolyphenylene polyisocyanate, a mixture of methylenediphenylene diisocyanate and polymethylenepolyphenylene polyisocyanate, 1,5-pentane diisocyanate, 1,6-hexamethylene diisocyanate, isophorone diisocyanate, xylylene diisocyanate or a mixture of two or more of the abovementioned isocyanates.

In a fourth embodiment of the invention, which is a particular configuration of the third embodiment, the isocyanate component comprises tolylene diisocyanate, methylenediphenylene diisocyanate, polymethylenepolyphenylene polyisocyanate or a mixture of two or more of the abovementioned isocyanates.

In a fifth embodiment of the invention, which is a particular configuration of the fourth embodiment, the isocyanate component comprises methylenediphenylene diisocyanate, polymethylenepolyphenylene polyisocyanate or—preferably—a mixture of both.

In a sixth embodiment of the invention, which is a particular configuration of the fifth embodiment, the isocyanate component comprises no further isocyanates.

In a seventh embodiment of the invention, which is a particular configuration of the fifth and sixth embodiment and may moreover be combined with all other embodiments, but in particular the first embodiment, the polyol component comprises a polyether polyol.

In an eighth embodiment of the invention, which is a particular configuration of the seventh embodiment, the number-average molar mass Mₙ of the polyether polyol is 400 g/mol to 1500 g/mol, preferably 400 g/mol to 1000 g/mol, and the hydroxyl functionality thereof is 2 to 8, preferably 2 to 6.

In a ninth embodiment of the invention, which may be combined with all other embodiments, the araliphatic monofunctional alcohol is benzyl alcohol, furfuryl alcohol or a mixture of both alcohols and is preferably benzyl alcohol.

In a tenth embodiment of the invention, which may be combined with all other embodiments, the alcoholysis catalyst comprises one or more of the following compounds:
a tin catalyst, for example a tin(II) biscarboxylate or a tin(IV) tetracarboxylate (in particular tin diacetate, tin dioctoate or tin tetraacetate), an organometallic tin carboxylate (in particular dibutyltin diacetate, dibutyltin dioctoate, dibutyltin dilaurate, dibutyltin benzylmaleate, diphenyltin diacetate or dibutyltin dioleate), an organometallic tin acetylacetonate (such as in particular dibutyltin diacetylacetonate), an organometallic tin alkoxide (such as in particular dimethoxydibutyltin), an organometallic tin silicate (such as in particular bis(triethoxysiloxy)dibutyltin) or a bis-organometallic tin(II) compound (such as in particular dimethyltin),
a titanium catalyst, for example a titanium alkoxylate (such as in particular tetraethoxytitanium, tetra-n-propoxytitanium, tetra-i-propoxytitanium, tetra-n-butoxytitanium, tetra-i-butoxytitanium, tetrakis(2-ethylhexoxy)titanium, a titanium acetylacetonate (such as in particular di-i-propoxybis(ethylaceto-acetate)titanium, dipropoxybis(acetylacetonate)titanium, di-i-propoxybis-(acetylacetonate)titanium, dibutoxybis(acetylacetonate)titanium or bis(acetylacetonate)titanium oxide) or isopropoxyoctylene glycol titanium oxide,
a lead catalyst, for example a lead acetate (such as in particular lead diacetate, lead di-2-ethylhexanoate, lead dineodecanoate, lead tetraacetate, lead tetrapropionate),
a zinc catalyst such as for example a zinc acetylacetonate (such as in particular zinc acetylacetonate) or a zinc acetate (such as in particular zinc 2-ethylcaproate, zinc diacetate, zinc dineodecanoate, zinc diundecenoate, zinc dimethacrylate),
a zirconium catalyst such as for example zirconium octoate or zirconium ethyl acetate complex,
a cobalt catalyst such as for example cobalt diacetate,
a bismuth catalyst, such as for example bismuth carboxylates (such as for example bismuth (III) neodecanoate) or bismuth oxide,
an iron catalyst such as for example iron (III) acetylacetonate or iron diacetate,
an aluminum catalyst, such as for example aluminum acetylacetonate,
a calcium catalyst such as for example calcium ethylenediamine tetraacetate,
a magnesium catalyst, for example magnesium ethylenediaminetetraacetate,
an organic amine such as for example triethylamine, tributylamine, aminopropyltrimethoxysilane, N-(2-aminoethyl)-3-aminopropyltrimethoxysilane, tetramethylguanidine, 1,8-diazabicyclo[5.4.0.]-7-undecene, 1,4-diazabicyclo[2.2.2]octane, 1,8-diazabicyclo[5.4.0]undec-7-ene, 1,5-diazabicyclo-[4.3.0]non-5-ene, N,N-bis(N,N-dimethyl-2-aminoethyl)methylamine, N,N-dimethylcyclohexylamine, N,N-dimethyl-phenylamine or N-ethylmorpholine,
a tetraalkylammonium compound, such as for example N,N, N-trimethyl-N-2-hydroxypropylammonium hydroxide, N,N,N-trimethyl-N-2-hydroxypropylammonium 2-ethylhexanoate or choline 2-ethylhexanoate.
an (organic or inorganic) Brønsted acid or esters thereof such as for example methanesulfonic acid, p-toluenesulfonic acid, dodecylbenzenesulfonic acid, 1-naphthalenesulfonic acid, camphorsulfonic acid, acetic acid, trifluoroacetic acid, hydrochloric acid, phosphoric acid, butyl phosphate, (iso) propyl phosphate or dibutyl phosphate
an (organic or inorganic) Brønsted base such as for example hydroxides (in particular sodium hydroxide, tetramethylammonium hydroxide, potassium hydroxide or tetrabutylammonium hydroxide, carbonates (in particular alkali metal carbonates such as for example sodium or potassium carbonate) or hydrogencarbonates (such as in particular alkali metal hydrogencarbonates such as for example sodium or potassium hydrogencarbonate) or
an acid halide such as for example benzoyl chloride.

In an eleventh embodiment of the invention, which may be combined with all other embodiments, step (B) is performed at a temperature in the range from 130° C. to 195° C., preferably 135° C. to 190° C., particularly preferably 140° C. to 190° C., very particularly preferably 165° C. to 185° C.

In a twelfth embodiment of the invention, which may be combined with all other embodiments, step (B) is performed at a pressure in the range from 900 mbar (abs.) to 1800 bar (abs.), in particular at ambient pressure.

In a thirteenth embodiment of the invention, which may be combined with all other embodiments, the monofunctional araliphatic alcohol and the polyurethane product are employed in a mass ratio
[m(monofunctional araliphatic alcohol)/m(polyurethane product)]
   in the range from 0.30 to 10, preferably 0.40 to 7.5, particularly preferably 0.45 to 5.0 and very particularly preferably 0.48 to 2.0 in step (B).

In a fourteenth embodiment of the invention, which may be combined with all other embodiments, the reaction in step (B) is performed for a period of 1.0 h to 10 h, preferably 1.5 h to 7.5 h, particularly preferably 2.0 h to 6.0 h and very particularly preferably 2.5 h to 5.5 h.

In an fifteenth embodiment of the invention, which may be combined with all other embodiments, step (C) is followed by the following:
(D) further reaction of the carbamate separated in (C), wherein the further reaction comprises one of the following:
(D.I) hydrolysis of the carbamate with water in the presence of a hydrolysis catalyst to form an amine corresponding to an isocyanate of the isocyanate component;
(D.II) cleavage of the carbamate into the isocyanate of the isocyanate component and the monofunctional araliphatic alcohol;
or
(D.III) hydrogenolysis of the carbamate with hydrogen to form an amine corresponding to an isocyanate of the isocyanate component in the presence of a hydrogenolysis catalyst.

In a sixteenth embodiment of the invention, which is a particular configuration of the fifteenth embodiment, the process comprises the step (D.I).

In a seventeenth embodiment of the invention, which is a particular configuration of the sixteenth embodiment, the hydrolysis catalyst comprises an (organic or inorganic) Brønsted base such as for example a hydroxide (in particular sodium hydroxide, tetramethylammonium hydroxide, potassium hydroxide or tetrabutylammonium hydroxide), a carbonate (in particular an alkali metal carbonate such as for example sodium or potassium carbonate) or a hydrogencarbonate (in particular an alkali metal hydrogencarbonate such as sodium or potassium hydrogencarbonate).

In an eighteenth embodiment of the invention, which is a particular configuration of the seventeenth embodiment, the water and the carbamate are employed in a mass ratio [m(water)/m(carbamate)] in the range from 0.05 to 2.5, preferably 1.3 to 1.7, for example 1.5.

In a nineteenth embodiment of the invention, which is a further particular configuration of the sixteenth embodiment, the hydrolysis catalyst is a urethanase.

In a twentieth embodiment of the invention, which is a particular configuration of the nineteenth embodiment, a urethanase is employed which is selected from the group consisting of SEQ ID no. 3, SEQ ID no. 4, SEQ ID no. 10, SEQ ID no. 11 (see WO 2019/243293 A1), preferably SEQ ID no. 3, SEQ ID no. 4, SEQ ID no. 11, particularly preferably SEQ ID no. 4 and SEQ ID no. 11, very particularly preferably SEQ ID no. 4, and variants of these polypeptides, wherein the abovementioned polypeptides exhibit urethanase activity.

In a twenty-first embodiment of the invention, which is a further particular configuration of the fifteenth embodiment, the process comprises the step (D.II), wherein the cleavage of the carbamate is performed without addition of a carbamate cleavage catalyst.

In a twenty-second embodiment of the invention, which is a further particular configuration of the fifteenth embodiment, the process comprises the step (D.II), wherein the cleavage of the carbamate is performed in the presence of a carbamate cleavage catalyst.

In a twenty-third embodiment of the invention, which is a particular configuration of the twenty-second embodiment, the carbamate cleavage catalyst comprises
a metal-free or metal-containing Brønsted- or Lewis-acidic catalyst
or
a metal-free or metal-containing Brønsted- or Lewis-basic catalyst.

In a twenty-fourth embodiment of the invention, which is a particular configuration of the twenty-first to twenty-third embodiment, step (D.II) is performed at a temperature in the range from 150° C. to 280° C. and at a pressure in the range from 0.01 bar (abs.) to 2.00 bar (abs.).

In a twenty-fifth embodiment of the invention, which is a further particular configuration of the fifteenth embodiment, the process comprises the step (D.III).

In a twenty-sixth embodiment of the invention, which is a particular configuration of the twenty-fifth embodiment, the hydrogenolysis is performed in the presence of a solvent (such as in particular methanol or ethanol) at a temperature in the range from 20° C. to 100° C.

In a twenty-seventh embodiment of the invention, which is a particular configuration of the twenty-fifth and twenty-sixth embodiment, the hydrogenolysis catalyst comprises palladium (in particular Pd/C, PdCl₂ or Pd(OAc)₂), nickel (in particular Raney nickel) or platinum (in particular platinum (IV) oxide).

In a twenty-eighth embodiment, which may be combined with all other embodiments, the process comprises the step
(E) obtaining polyols from the polyol phase.

In a twenty-ninth embodiment of the invention, which is a particular configuration of the twenty-eighth embodiment, step (E) comprises a distillation, a stripping with a stripping gas (such as especially nitrogen or steam, preferably nitrogen) or a combination of these measures.

The embodiments outlined briefly above and further possible configurations of the invention are more particularly elucidated hereinbelow. All embodiments and further possible configurations of the invention may be combined with one another as desired, unless expressly stated otherwise or unless the opposite is clearly evident to a person skilled in the art from the context.

### Polyurethane Products

The present invention is in principle applicable to the polyurethane products known in the art. These are based on the abovementioned polyol and isocyanate components, wherein polyether polyols and mMDI, pMDI or MDI, optionally in admixture with other isocyanates such as in particular TDI, are preferred. The polyurethane product is particularly preferably based on MDI as the sole isocyanate of the isocyanate component and on a polyether polyol as the sole polyol of the polyol component. As already elucidated hereinabove this does not exclude the possibility of the use of mixtures of different polyether polyol types (and naturally not of mixtures of different MDI types either).

The invention is particularly suitable for the recycling of polyurethane foams, in particular rigid polyurethane foams. A rigid polyurethane foam is a highly crosslinked thermoset plastic which is foamed to afford a cellular construct of low bulk density (in particular in the range from 30 kg/m³ to 90 kg/m³, preferably 30 kg/m³ to 45 kg/m³; determined according to DIN EN ISO 845:2009-10 and low thermal conductivity (generally in the range from 0.021 W/(m·K) to 0.030 W/(m·K) determined according to DIN 52612 part 2:1984-06-01). It is generally closed-celled and exhibits a relatively high deformation resistance under compressive stress. The thermoset character is reflected in the fact that the foam is not fusible and has a high softening point and good resistance to chemicals and solvents. Rigid polyurethane foams especially have a compressive strength at 40% determined according to DIN EN ISO 604:2003 of 200 kPa to 700 kPa and a tensile strength determined according to DIN EN 826:2013-05-01 of 200 kPa to 900 kPa.

Rigid polyurethane foams are generally produced using comparatively short-chain polyols, in particular short-chain polyether polyols. The short-chain polyether polyols are preferably based on sugar starters (for example sucrose or sorbitol) in particular with admixtures of glycols (for example ethylene glycol or propylene glycol) or aromatic amines (for example tolylenediamine, in particular the 2,4-isomer) as further starters. They preferably have a (theoretical, i.e. corresponding to the starter used) hydroxyl functionality of 2 to 8, preferably 2 to 6, and a number-average molar mass Mₙ in the range from 400 g/mol to 1500 g/mol, preferably 400 g/mol to 1000 g/mol. In the context of the present invention the determination of molar masses is carried out by gel permeation chromatography (GPC). The following measurement conditions were adhered to when determining the mass-average molar mass Mₙ and the polydispersity M_{w}/Mₙ:
column combination SDV 1000/10000 Å (length 65 cm), temperature 30° C., THF mobile phase, flow rate 1 ml/min, sample concentration 10 g/l, RI detector. Evaluation of the polyether polyols was carried out against polystyrene standard (162 g/mol to 2.57×10⁴ g/mol). The OH numbers of such polyether polyols are in the range from 100 mgKOH/g to 600 mgKOH/g.

When mixtures of different polyether polyols are employed the abovementioned values for molar mass and hydroxyl functionality apply to each polyether polyol which is a constituent of the mixture so that the values averaged over all polyether polyols of the mixture are also within the recited ranges.

### Preparation of the Chemical Recycling

The above-described polyurethane products are provided for the subsequent transurethanization in step (A). It is preferable when this step (A) already comprises preparatory steps for the cleavage of the urethane bonds in step (B). These are especially mechanical comminution of the polyurethane products. Such preparatory steps are known to a person skilled in the art; reference is made by way of example to the literature cited in [1]. Depending on the constitution of the polyurethane product it may be advantageous to “freeze” it before the mechanical comminution in order to facilitate the comminuting operation. This applies especially to the polyurethane foams preferably to be reacted in step (B).

Before, during or after the mechanical comminution the polyurethane product may be subjected to treatment with aqueous or alcoholic disinfectants. Such disinfectants are preferably hydrogen peroxide, chlorine dioxide, sodium hypochlorite, formaldehyde, sodium N-chloro-(4-methylbenzene) sulfonamide (chloramine T) and/or peracetic acid (aqueous disinfectants) or ethanol, isopropanol and/or 1-propanol (alcoholic disinfectants).

It is also conceivable to perform the above-described preparatory steps at a location spatially separate from the location of the chemolysis in step (B), in particular in a different production site. In that case, the prepared polyurethane product is transferred into suitable transport vehicles, for example silo vehicles, for further transport. For further transport the prepared polyurethane product may additionally be compressed to achieve a higher mass-to-volume ratio. The polyurethane product is then transferred into the reaction apparatus provided for the chemolysis at the location of the chemolysis. It is also conceivable to connect the employed transport vehicle directly to the reaction apparatuses.

### Chemolysis of the Polyurethane Product

Step (B) of the process according to the invention includes the chemolysis (=transurethanization) of the polyurethane product provided in step (A).

The chemolysis is preferably carried out in the absence of oxygen. This is to be understood as meaning that the reaction is carried out in an inert gas atmosphere (especially in a nitrogen, argon or helium atmosphere). It is also preferable to free the employed chemolysis reagents of oxygen by inert gas saturation.

Suitable monofunctional aliphatic alcohols include in particular benzyl alcohol, furfuryl alcohol or a mixture of both alcohols. Benzyl alcohol is preferred. It is particularly preferable not to employ any further reactants capable of cleaving urethane bonds in addition to the monofunctional araliphatic alcohol. In the context of the present invention amounts of water (altogether up to 5%, in particular up to 2% of the mass of the altogether employed monofunctional araliphatic alcohol) which may for example derive from the polyurethane product, be dissolved in the alcohol or be employed as solvent for the alcoholysis catalysis are to be understood not as reactants capable of cleaving urethane bonds but rather as trace constituents having an immaterial effect on the reaction.

Suitable alcoholysis catalysts include in particular tin catalysts, titanium catalyst, lead catalysts, zinc catalysts, zirconium catalysts, cobalt catalysts, bismuth catalysts, iron catalysts, aluminum catalysts, calcium catalysts, magnesium catalysts, organic amines, tetraalkyl ammonium compounds, (organic or inorganic) Brønsted acids or esters thereof, (organic or inorganic) Brønsted bases or acid halides. Preferred representatives of such catalysts are described hereinabove.

The temperature for step (B) is preferably in the range from 130° C. to 195° C., particularly preferably from 135° C. to 190° C., very particularly preferably from 140° C. to 190° C. and exceptionally preferably from 165° C. to 185° C. The pressure in step (B) is preferably in the range from 900 mbar (abs.) to 1800 mbar (abs.) and in particular corresponds to atmospheric pressure.

In step (B) the monofunctional aliphatic alcohol and the polyurethane product are preferably employed in a mass ratio [m(monofunctional aralaliphatic alcohol)/m(polyurethane product)] in the range from 0.30 to 10, particularly preferably 0.40 to 7.5, very particularly preferably 0.45 to 5.0 and exceptionally preferably 0.48 to 2.0. The total reaction duration is preferably 1.0 h to 10 h, particularly preferably 1.5 h to 7.5 h, very particularly preferably 2.0 h to 6.0 h and exceptionally preferably 2.5 h to 5.5 h.

### Isolation of the Carbamate

In the subsequent step (C) the carbamate is isolated from the suspension obtained in step (B) which—since the carbamate is obtained as a solid in the context of the present invention—is very easily achievable by filtration or centrifugation. Depending on the manner of the further use of the carbamate this may be subjected to further workup, for example washed or recrystallized.

### Further Processing of the Carbamate

The carbamate obtained in step (C) may be supplied to various intended uses in a step (D). These include in particular the following further possible reactions:
The carbamate may be subjected to a catalytic hydrolysis with water to form an amine corresponding to an isocyanate of the isocyanate component [(D.I)]. The resulting amines may then be supplied to numerous further applications, inter alia may be phosgenated to re-form the corresponding isocyanate. The isocyanate thus obtained may then be returned to the production of polyurethane products.
However, it is also conceivable to obtain the isocyanate of the isocyanate component directly from the carbamate by cleaving [(D.II)] the carbamate into the isocyanate of the isocyanate component and the monofunctional araliphatic alcohol either by purely thermal means or in the presence of a carbamate cleavage catalyst. The resulting monofunctional araliphatic alcohol may be reused in step (B) after separation from the isocyanate while the isocyanate may be returned to the production of polyurethane products.
The carbamate may finally also be subjected to a catalytic hydrogenolysis with hydrogen to form an amine corresponding to an isocyanate of the isocyanate component [(D.III)]. The amine may also be further used as described at (D.I).

Reactions according to (D.I) to (D.III) are known per se from the literature and therefore only outlined briefly below.

Both chemical and biological (=enzymatic) catalysts are suitable for the catalytic hydrolysis with water [(D.I)].

Contemplated chemical hydrolysis catalysts especially include (organic or inorganic) Brønsted bases such as for example hydroxides (in particular sodium hydroxide, tetramethylammonium hydroxide, potassium hydroxide or tetrabutylammonium hydroxide, carbonates (in particular an alkali metal carbonate such as for example sodium or potassium carbonate) or hydrogencarbonates (in particular an alkali metal hydrogen carbonate such as sodium or potassium hydrogencarbonate). The mass ratio of water and carbamate [m(water)/m(carbamate)] is preferably in the range from 0.05 to 2.5, particularly preferably 1.3 to 1.7, for example 1.5.

Suitable enzymatic hydrolysis catalysts especially include urethanases, i.e. enzymes which cleave a urethane bond to liberate one mol of amine, one mol of alcohol and one mol of CO₂ per urethane group. The liberated amine corresponds to the amine from which the isocyanate used for synthesizing the polyurethane product may be produced by phosgenation.

Preferred urethanases are selected from the group consisting of SEQ ID no. 3, SEQ ID no. 4, SEQ ID no. 10, SEQ ID no. 11, preferably SEQ ID no. 3, SEQ ID no. 4, SEQ ID no. 11, particularly preferably SEQ ID no. 4 and SEQ ID no. 11, very particularly preferably SEQ ID no. 4, and variants of these polypeptides, characterized in that the abovementioned polypeptides exhibit urethanase activity. The amino acid sequences of the abovementioned enzymes are also disclosed under the same designations in WO 2019/243293 A1.

The term “polypeptide” is well known to those skilled in the art. It refers to a chain of at least 50, preferably at least 70, amino acids linked to one another by peptide linkages. A polypeptide may comprise both naturally occurring and synthetic amino acids. It preferably comprises the known proteinogenic amino acids.

A “variant” is obtained by adding, deleting or replacing up to 10%, preferably up to 5%, of the amino acids present in the respective polypeptide. Particularly preferred variants of the abovementioned polypeptides are obtained by adding, deleting or exchanging up to 20, preferably up to 10, and more preferably up to 5, amino acids of the disclosed sequences. The abovementioned modifications may in principle be executed continuously or discontinuously at any desired point in the polypeptide. However, they are preferably executed only at the N-terminus and/or at the C-terminus of the polypeptide. Each variant obtained by adding, replacing or deleting amino acids according to the invention is, however, characterized by urethanase activity as defined in this application hereinbelow.

The term “urethanase activity” refers to the ability of a polypeptide to enzymatically catalyze the cleavage of a urethane group. In this process, each mole of urethane group gives rise to one mole of amine, one mole of alcohol, and one mole of CO 2.

The expression “enzymatic cleavage of a urethane group” indicates that the cleavage of a urethane group described above proceeds more rapidly in the presence of a polypeptide having urethanase activity than it does when incubated with the reaction buffer without enzyme under the same reaction conditions or when incubated with the reaction buffer under the same conditions in the presence of an inactive polypeptide. The preferred model for an inactive polypeptide is bovine serum albumin. If, in the presence of a polypeptide being tested, the cleavage of the urethane group proceeds more rapidly than in an otherwise identical control with BSA, said polypeptide possesses urethane activity as understood in this application.

The urethane group may be an aromatically or aliphatically attached urethane group. In the case of an aromatically attached urethane group, the nitrogen atom is attached directly to an aromatic ring. In the case of an aliphatically attached urethane group, the nitrogen atom is attached to an alkyl radical. The alkyl radical is preferably unbranched and composed of at least one, preferably at least two, and more preferably at least three, carbon atoms. In a preferred embodiment of the present invention the polypeptide having urethanase activity is capable of enzymatically cleaving an aromatically bonded urethane group.

Whether a polypeptide has urethanase activity can be checked through the cleavage of suitable model substrates.

It is preferable to employ ethyl-4-nitrophenylcarbamate (ENPC) as a model substrate for investigating the hydrolysis ability of carbamates having an aromatic radical bonded to their nitrogen atom. Cleavage is demonstrated by determining the increase in the concentration of 4-nitroaniline. This is done preferably photometrically at a wavelength of 405 nm. The enzyme activity is determined preferably in a reaction buffer containing 100 mM K₂HPO₄/KH₂PO₄, PH 7 with 6.25% by volume of ethanol in the presence of 0.2 mg/L of ENPC as substrate. Incubation of the enzyme with ENPC in the reaction buffer is carried out preferably at room temperature and preferably for 24 hours.

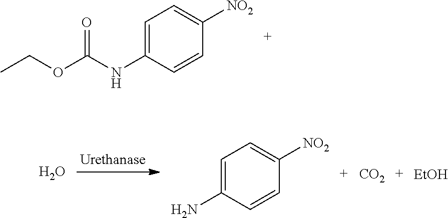

It is preferable to employ ethylphenethyl carbamate (EPEC) as a model substrate for investigating the hydrolysis ability of carbamates having an aliphatic radical bonded to their nitrogen atom. Cleavage is demonstrated by determining the increase in the concentration of phenethylamine. This is done preferably by HPLC. The reaction buffer used and the reaction conditions preferably correspond to the parameters described above for ENPC.

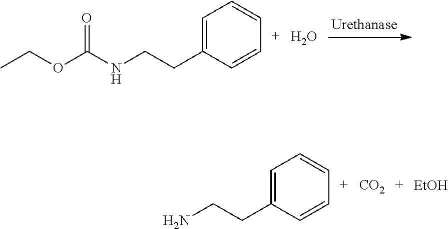

Whether a polypeptide with urethanase activity from WO 2019/243293 A1 is suitable for hydrolysis of MDI carbamates deriving from the alcoholysis of an MDI-based polyurethane product with a monofunctional araliphatic alcohol, in particular benzyl alcohol, may be determined by direct detection of the product by HPLC. This detects liberation of mMDA or pMDA. Screening is performed using a model carbamate formed from pMDI and benzyl alcohol. This is abbreviated to pMDI-benzyl alcohol hereinbelow. The temperature of the enzymatic reaction may preferably be in a range from 20° C. to 70° C. The reaction is preferably carried out at ambient pressure. The solvent used for the enzymatic invention is preferably phosphate buffer. DMSO (dimethylsulfoxide) is preferably employed as a cosolvent for the carbamates. A corresponding approach may be taken for other polyurethane products.

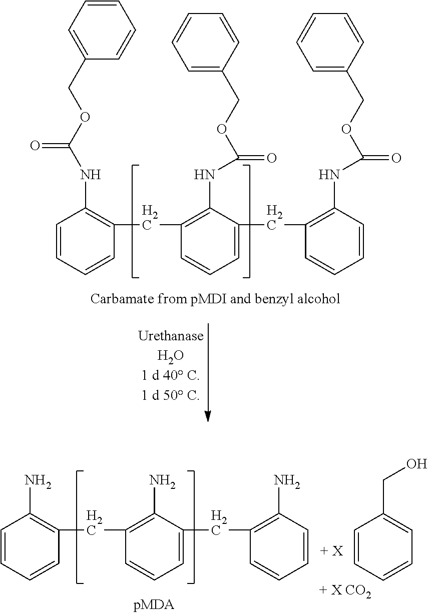

The carbamate cleavage reaction [(D.II)], the thermal, optionally catalytically-assisted cleavage of the carbamate into the isocyanate of the isocyanate component and the monofunctional araliphatic alcohol, may be performed at temperatures in the range from 150° C. to 280° C. and at pressures in the range from 0.001 bar (abs.) to 2.00 bar (abs.). When the reaction is catalyzed suitable carbamate cleavage catalysts include in particular
metal-free or metal-containing Brønsted- or Lewis-acidic catalysts
or
metal-free or metal-containing Brønsted- or Lewis-basic
   catalysts.

The catalytic hydrogenolysis with hydrogen [(D.III)] is preferably performed in the presence of a solvent (such as in particular methanol or ethanol) at a temperature in the range from 20° C. to 100° C. Suitable hydrogenolysis catalysts include in particular palladium (especially Pd/C, PdCl₂ or Pd(OAc)₂), nickel (especially Raney nickel) or platinum catalysts (especially platinum (IV) oxide). Performing step (D) as a hydrogenolysis has the particular advantage that certain byproducts (such as in particular N-benzyl compounds) that may be formed react to form easily separable subsequent products (especially toluene).

### Further Processing of the Polyol Phase

It goes without saying that the polyol phase obtained in step (C) also constitutes a value product which is preferably further used. To this end the polyols present therein are isolated/purified in a step (E). Step (E) preferably comprises a distillation, a stripping with a stripping gas (such as especially nitrogen or steam, preferably nitrogen) or a combination of both methods. The polyols recovered in this way may be returned to the production of polyurethane products.

### EXAMPLES

### A. Examples for Alcoholysis of an MDI-Based Rigid Foam

### General Comments

The following examples 1 to 6 were performed with benzyl alcohol and the comparative examples 7 to 8 with ethanol/phenylethanol as monofunctional alcohol. In all examples an MDI-based rigid polyurethane foam was used as the polyurethane product to be cleaved. The MDI-based carbamate compounds corresponding to the employed alcohols were analyzed by LC-MS. The method employed was as follows:

5-95% of water-acetonitrile (+0.1% formic acid) gradient on an Agilent “EC-C18 50×3 mm 2.7 μm dp” separating column at 35° C. and a flow rate of 0.8 ml/min. Recording of positive and negative mass spectra and tandem mass spectra at a resolution of 35000 Th at m/z 200 Th. Th is short for the unit Thomson which describes the mass-charge ratio m/z.

The rigid polyurethane foam was produced according to the following formulation.

TABLE 1
Formulation of the polyurethane foam cleaved in the
examples (in parts by weight)

| | |
|---|---|
| Desmophen 21AP17 (1) | 41.71 |
| Tegostab BF 8465 (2) | 0.43 |
| Desmorapid PV (3) | 0.09 |
| Desmorapid 726 B (4) | 0.43 |
| Water | 0.65 |
| Cyclopentane (5) | 4.34 |
| Desmodur 44V20L (6) | 52.34 |
| Index (7) | 110 |

Key:
(1) Polyether polyol from Covestro Deutschland AG
(2) Polyether siloxane additive from Evonik AG
(3) Desmorapid PV is an amine catalyst from Covestro Deutschland AG
(4) Desmorapid 726 B is an amine catalyst from Covestro Deutschland AG
(5) Cyclopentane is a physical blowing agent
(6) Desmodur 44V20L is an MDI from Covestro Deutschland AG
(7) Ratio of NCO to OH groups

### General Experimental Procedure

In a 1000 mL 4-necked flask fitted with a stirrer, thermometer and cooler, alcohol and catalyst are initially charged and heated to 180° C. to 190° C. under nitrogen. A rigid foam produced according to the formulation from table 1 is added at this temperature and after dissolution thereof the resulting mixture is stirred at 180° C. to 190° C. for 3 hours. Table 2 shows the conditions and the results achieved.

TABLE 2
Results of examples

| | Ex. 1 | Ex. 2 | Ex. 3 | Ex. 4 | Ex. 5 | Ex. 6 | Ex. 7 | Ex. 8 |
|---|---|---|---|---|---|---|---|---|
| | (inv.) | (inv.) | (inv.) | (inv.) | (inv.) | (inv.) | (comp.) | (comp.) |
| m(benzyl | 300 | 300 | 300 | 300 | 900 | 141 | | |
| alcohol)/g | | | | | | | | |
| m(ethanol)/g | | | | | | | 60 | |
| m(phenyl- | | | | | | | | 300 |
| ethanol)/g | | | | | | | | |
| m(DBU)/g | 0.9 | | | | | | | |
| m(DABCO)/g | | 0.9 | | | | | | |
| m(Na₂CO₃)/g | | | | | 12.15 | | | |
| m(tetrabutyl | | | 0.23 | 0.3 | | 0.22 | 0.18 | 0.3 |
| titanate)/g | | | | | | | | |
| Rigid foam/g | 150 | 150 | 150 | 300 | 450 | 300 | 300 | 300 |
| 1,2-dichloro- | | | | | | 400 | 400 | |
| benzene/g | | | | | | | | |
| Yield of MDI | 18.4 | 18.4 | 29.0 | 40.0 | 84.0 | 38.8 | — | — |
| biscarbamate/g | | | | | | | | |

Key: inv. = inventive; DBU = 1,8-diazabicyclo[5.4.0]undec-7-ene; DABCO = 1,4-diazabicyclo[2.2.2]octane.
(Methylenedi-p-phenylene)bis(phenylmethylcarbamate) (examples 1 to 6) having a molar mass of 466.5 g/mol was in each case demonstrated as the main product by LC-MS. (The polymeric carbamates from pMDI were likewise demonstrated but not quantified).

In comparative example 7 the rigid polyurethane foam was not dissolvable. In comparative example 8 the MDA-phenylethanol carbamate obtained was not solid but viscous and was therefore inseparable from the reaction mixture and from the polyol.

### B. Examples for Enzymatic Hydrolysis of MDI Carbamates

### General Comments and Experimental Procedure

The following examples employed urethanases from WO 2019/243293 A1. The enzymes were expressed in Escherichia coli BL21 (DE3), digested and lyophilized. 0.9% (w/w) of this preparation was admixed with 1% (w/w) of substrate (here carbamates) in phosphate buffer (preferably between 50 to 200 mM, pH 7.5). Dimethyl sulfoxide is employed as cosolvent for the substrate (not more than 10% (w/w) of the final reaction). An esterase from Sigma Aldrich was also investigated: PLE (esterase from porcine liver (E2884-5kU)) (SEQ ID no. 11, WO 2019/243293). The reaction is carried out for 24 h at 40° C. and a further 24 h at 50° C. in 1.5 mL reaction vessels in a Thermoblock at 1000 rpm. The enzymatic reaction is stopped by diluting the reaction batch in acetonitrile or stopping solution (50 mM NaOH, 20% acetic acid, 50% acetonitrile). The samples are filtered and analyzed by HPLC (ZORBAX Eclipse C18 column (particle size 3.5 μm, 4.6×75 mm (Agilent Technologies, Santa Clara, USA), 40° C., eluent A: acetonitrile with 5% ultrapure water, eluent B: 10 mM sodium phosphate buffer pH 7.0 with 5% ACN, flow: 1 mL/min). The profile of the HPLC method is shown in table 3. Table 4 shows the elution times of the components with this measurement method.

TABLE 3
Profile of HPLC method

| Time/ | | | Flow/ | Maximum allowable |
|---|---|---|---|---|
| min | A % | B % | ml · min⁻¹ | pressure/bar |
| 0.00 | 0 | 100 | 1 | 300 |
| 2.00 | 0 | 100 | 1 | 300 |
| 10.00 | 95 | 5 | 1 | 300 |
| 11.00 | 95 | 5 | 1 | 300 |
| 11.50 | 0 | 100 | 1 | 300 |
| 17.00 | 0 | 100 | 1 | 300 |

TABLE 4
Elution times in HPLC analysis for the
employed carbamate pMDI-benzyl alcohol and the
expected products 4,4′-MDA and pMDA.

| Component | Elution time [min] |
|---|---|
| Benzyl alcohol | 7.3 |
| 4,4′-MDA | 8.4 |
| Regioisomers and polymers of MDA (pMDA) | between 8.8-9.4 |

### Examples 9 to 12

As is apparent from table 5 which follows, liberation of 4,4′-MDA and pMDA from pMDI-benzyl alcohol was identified in three enzymatic reactions: with Aes72 (SEQ ID no. 3, WO 2019/243293 A1), PLE (SEQ ID no. 11, WO 2019/243293 A1) and Aes170 (SEQ ID no. 4, WO 2019/243293). Liberation of benzyl alcohol was also demonstrated. These results were achievable both with synthetically produced carbamate and with carbamate from the benzyl alcoholysis from the preceding examples. The carbamate from example 1 and example 3 (table 2) served as starting material.

TABLE 5
Results of examples for enzymatic hydrolysis

| | | | a) hydrolysis of | |
|---|---|---|---|---|
| | | | pMDA-benzyl | |
| | | | alcohol | b) hydrolysis of |
| | | | (synthetically | the carbamate |
| | | | produced from | from the |
| | | | pMDI with | alcoholysis |
| | SEQ ID no. (WO | Designation | excess benzyl | with benzyl |
| Ex. | 2019/243293 A1) | in study | alcohol) | alcohol¹ |
| 9 | 3 | Aes72 | yes | yes |
| 10 | 11 | PLE | yes² | yes² |
| 11 | 4 | Aes170 | yes² | yes² |
| 12 | 10 | AesGö56 | partial | partial |

¹Examples 9b to 12b were each performed once with the carbamate from example 1 and once with the carbamate from example 3. The results were identical.
²In addition to 4,4′-MDA the liberation of benzyl alcohol was also detected.

1. A process for cleavage of polyurethane products comprising:
(A) providing a polyurethane product based on an isocyanate component and a polyol component;
(B) reacting the polyurethane product with a monofunctional araliphatic alcohol in the presence of an alcoholysis catalyst to obtain a product mixture containing a liquid polyol phase and a solid carbamate of an isocyanate of the isocyanate component and of the monofunctional araliphatic alcohol;
And
(C) separating the carbamate from the product mixture, leaving behind the liquid polyol phase.

2. The process as claimed in claim 1, wherein the polyurethane product comprises a polyurethane foam.

3. The process as claimed in claim 1, wherein the polyol component comprises a polyether polyol, a polyester polyol, a polyether ester polyol, a polyether carbonate polyol, a polyacrylate polyol or a mixture of two or more of the abovementioned polyols.

4. The process as claimed in claim 1, wherein the isocyanate component comprises tolylene diisocyanate, methylenediphenylene diisocyanate, polymethylenepolyphenylene polyisocyanate, a mixture of methylenediphenylene diisocyanate and polymethylenepolyphenylene polyisocyanate, 1,5-pentane diisocyanate, 1,6-hexamethylene diisocyanate, isophorone diisocyanate, xylylene diisocyanate or a mixture of two or more of the abovementioned isocyanates.

5. The process as claimed in claim 1, wherein the araliphatic monofunctional alcohol comprises benzyl alcohol, furfuryl alcohol or a mixture of both alcohols.

6. The process as claimed in claim 1, wherein step (B) is performed at a temperature in the range from 130° C. to 195° C.

7. The process as claimed in claim 1, wherein the monofunctional araliphatic alcohol and the polyurethane product are employed in a mass ratio
[m(monofunctional araliphatic alcohol)/m(polyurethane product)]
in the range from 0.30 to 10 in step (B).

8. The process as claimed in claim 1, wherein step (C) is followed by:
(D) further reaction of the carbamate separated in (C), wherein the further reaction comprises:
(D.I) hydrolysis of the carbamate with water in the presence of a hydrolysis catalyst to form an amine corresponding to an isocyanate of the isocyanate component;
(D.II) cleavage of the carbamate into the isocyanate of the isocyanate component and the monofunctional araliphatic alcohol;
or
(D.III) hydrogenolysis of the carbamate with hydrogen to form an amine corresponding to an isocyanate of the isocyanate component in the presence of a hydrogenolysis catalyst.

9. The process as claimed in claim 8 comprising step (D.I), wherein the hydrolysis catalyst comprises an organic or inorganic Brønsted base.

10. The process as claimed in claim 9, wherein the organic or inorganic Brønsted base comprises a hydroxide, a carbonate or a hydrogencarbonate.

11. The process as claimed in claim 8 comprising step (D.I), wherein the hydrolysis catalyst comprises a urethanase.

12. The process as claimed in claim 11, wherein the urethanase comprises a polypeptide comprising SEQ ID no. 3, SEQ ID no. 4, SEQ ID no. 10, SEQ ID no. 11 or a variant of any thereof, wherein the polypeptide exhibits urethanase activity.

13. The process as claimed in claim 8, comprising step (D.II), wherein step (D.II) is performed at a temperature in the range from 150° C. to 280° C. and at a pressure in the range from 0.001 bar_{(abs.)} to 2.00 bar_{(abs.)}.

14. The process as claimed in claim 8, comprising step (D.III), wherein and the hydrogenolysis is performed in the presence of a solvent at a temperature in the range from 20° C. to 100° C.

15. The process as claimed in claim 1, further comprising:
(E) obtaining polyols from the polyol phase.
